(19) United States
(12) Patent Application Publication
(10) Pub. No.: US 2021/0259535 A1
(43) Pub. Date: Aug. 26, 2021

(54) INTRAORAL IMAGING DEVICE

(71) Applicant: PLAQLESS LTD, Yokneam Ilit (IL)

(72) Inventors: Yuval Shani, Pardes Chana Karkur (IL); Tamir Ygal, Eschar (IL)

(21) Appl. No.: 17/256,712

(22) PCT Filed: Jul. 11, 2019
(86) PCT No.: PCT/IL2019/050787

§ 371 Date: Dec. 29, 2020

Related U.S. Application Data
(60) Provisional application No. 62/697,440, filed on Jul. 13, 2018.

(51) Int. Cl. A61B 1/24; A61B 1/05; A61B 1/06; A46B 5/00; A46B 9/04; A46B 15/00

(52) U.S. Cl. CPC A61B 1/24; A61B 1/05; A61B 1/0615; A46B 2200/1066; A46B 5/0095; A46B 9/04; A46B 15/0034; A61B 1/0676

(57) ABSTRACT

An intraoral imaging device comprises an elongated structural element; one or more light sources mounted on the structural element and configured to illuminate a target intraoral structure; one or more sensors mounted on the structural element and configured to detect the light returned from the illuminated target intraoral structure in order to generate an image; and a light absorbing divider dividing each of the light sources from an adjacent sensor and configured to obstruct direct emission of the light from the one or more light sources to the adjacent sensor.

### FIELD OF THE INVENTION

The present invention relates to the field of dental and oral health. More particularly, the invention relates to an intraoral imaging device.

### BACKGROUND OF THE INVENTION

The use of intraoral imaging devices has increased significantly in recent years, particularly by dental hygienists for educating patients about incorrect brushing procedures or periodontal disease, and also by dentists to produce a digital impression of a damaged tooth and of the position of an implant intended to replace the damaged tooth.

The intraoral imaging device of the present invention is a compact and inexpensive device that comprises one or more low-power light sources for illuminating an intraoral structure, and a sensor in close proximity to the light source, usually a solid-state detector such as a camera, for detecting the light returning from the target intraoral structure in order to generate an image. The target-facing optical plane of both the light sources and the sensor are configured to be substantially coplanar to prevent physical interference with other intraoral structures during an imaging operation as well as to optimize the intensity and uniformity of the light impinging upon the target intraoral structure.

Since the target-facing optical plane of both the light source and the sensor are positioned at a common level and do not protrude one from the other, the sensor receives not only light reflected from the target intraoral structure, but also light directly emitted from a light source, a condition which could cause saturation of the sensor. When saturated, the sensor will be unable to detect the reflected light with sufficient sensitivity to generate images that are representative of the target intraoral structure.

It is therefore an object of the present invention to provide an intraoral imaging device that prevents saturation of a light detecting sensor, even though the imaging device is configured with a light source and sensor which are in close proximity to each other and whose target-facing optical plane are substantially coplanar.

Other objects and advantages of the invention will become apparent as the description proceeds.

### SUMMARY OF THE INVENTION

An intraoral imaging device, comprising an elongated structural element; one or more light sources mounted on said structural element and configured to illuminate a target intraoral structure; one or more sensors mounted on said structural element and configured to detect the light returned from the illuminated target intraoral structure in order to generate an image; and a light absorbing divider dividing each of said light sources from an adjacent sensor and configured to obstruct direct emission of the light from said one or more light sources to said adjacent sensor.

In one aspect, the divider is opaque and is configured to completely block direct emission of the light from the one or more light sources to the adjacent sensor.

In one aspect, a target-facing optical plane of each of the light sources and of each of the sensors are substantially coplanar.

In one aspect, the intraoral imaging device may further comprise a window assembly which is configured with a plurality of protective transparent window elements for covering each of the light sources and sensors, each of said window elements constituting the target-facing optical plane, and a divider mount which is configured to position the divider in a void region between a first window element covering one of the light sources and a second window element which is adjacent to the first window element and covers one of the sensors.

In one aspect, the window assembly is formed with a thickened portion to ensure that the target-facing optical plane of each of the light sources and of each of the sensors are substantially coplanar even though a forward surface of one of the light sources and one of the sensors while mounted on the structural element is forwardly spaced from one another.

In one aspect, the window assembly is mounted on the structural element.

In one aspect, the intraoral imaging device further comprises an interchangeable head with which the window assembly is fitted and within which the structural element is insertable.

An additional divider dividing one of the light sources from the adjacent sensor may be mounted on the structural element. When the interchangeable head is coupled with a base member of the intraoral device, the structural element mounted divider may be aligned with the head mounted divider to prevent light emitted by one of the light sources from being reflected from the head mounted divider onto the sensor.

In one aspect, the interchangeable head is a toothbrush head having a toothbrush provided with at least some light absorbing bristles to prevent reflection of the light onto one of the sensors.

In one aspect, each of the light sources is mounted at a different lengthwise region of the structural element.

In one aspect, a distance between each of the light sources and the adjacent sensor ranges from 0.3 to 1.5 mm.

### BRIEF DESCRIPTION OF THE DRAWINGS

In the drawings:

FIG. 1 is a perspective view from the front of an embodiment of an imaging device comprising at least one light source and a sensor which is usable in conjunction with an intraoral device;

FIG. 2 is a perspective view from the front of a disassembled intraoral device, showing an exposed post on which are mounted the at least one light source and sensor of the imaging device of FIG. 1;

FIG. 3 is a perspective view from the front of an interchangeable head which is able to be coupled with the disassembled intraoral device of FIG. 2;

FIG. 4 is a perspective view from the front of an assembled intraoral device with which is coupled the interchangeable head of FIG. 3;

FIG. 5 is a perspective view of an upper portion of a toothbrush provided with the imaging device of FIG. 1, according to an embodiment;

FIG. 6 is a schematic cross sectional view of an upper portion of an intraoral device that includes an imaging device and a window assembly, according to an embodiment;

FIG. 7 is a perspective view from the front of a frame used in conjunction with the window assembly of FIG. 6, when separated from the intraoral device;

FIG. 8 is a perspective view from the front of a divider mount which cooperates with the frame of FIG. 7;

FIG. 9 is a side view of the divider mount of FIG. 8, when cooperating with the frame;

FIG. 10 is a perspective view from the front of a window assembly, according to another embodiment;

FIG. 11 is a perspective view from the front of a frame used in conjunction with the window assembly of FIG. 10;

FIG. 12 is a schematic illustration from the side of an imaging device, shown without a divider;

FIG. 13 is a schematic illustration from the side of an imaging device, shown with two dividers;

FIG. 14 is a schematic illustration of an imaging device configured with a divider whose width is less than the distance between each light source and sensor; and

FIG. 15 is a schematic illustration of an imaging device configured with a divider having a varying orientation.

### DETAILED DESCRIPTION OF THE INVENTION

A compact and inexpensive intraoral imaging device, which is preferably of light weight to maximize user control for both home use and use by a medical practitioner, comprises one or more light sources, one or more sensors and a window assembly having a frame, generally opaque, which divides each light source from an adjacent sensor and blocks the direct emission of light from a light source to an adjacent sensor when the divider is opaque. Each of the light sources may be a light-emitting diode (LED), a monochromatic light source, a fluorescing light source, an incoherent light source, or another type of light emitting unit. Each sensor may be an image sensor, such as a charge-coupled device (CCD), a complementary metal-oxide-semiconductor (CMOS), or another type of suitable sensor that detects and conveys information used to generate an image, such as one associated with a camera.

The frame of the window assembly may be configured with a thickened portion to ensure that the outer light emitting surface of the light source and the outer receiving surface of the sensor will be substantially coplanar, to prevent physical interference with other dental structures during an imaging operation as well as to optimize the intensity and uniformity of the light impinging upon the target intraoral structure, and its divider serves to obstruct or block the direct emission of light from a light source to an adjacent sensor, so that the direct emission does not saturate the sensor. Alternatively, the imaging device is factory fabricated in such a way to ensure that the outer light emitting surface of the light source and the outer receiving surface of the sensor will be substantially coplanar.

By maximizing uniformity of the light on the intraoral target, the sensor will be able to detect the reflected light with sufficient sensitivity to generate images that are representative of the target intraoral structure and that are able to be processed. If the light is not uniformly distributed, some regions of the intraoral target will not be properly illuminated or even subject to shadowing, and therefore unusable.

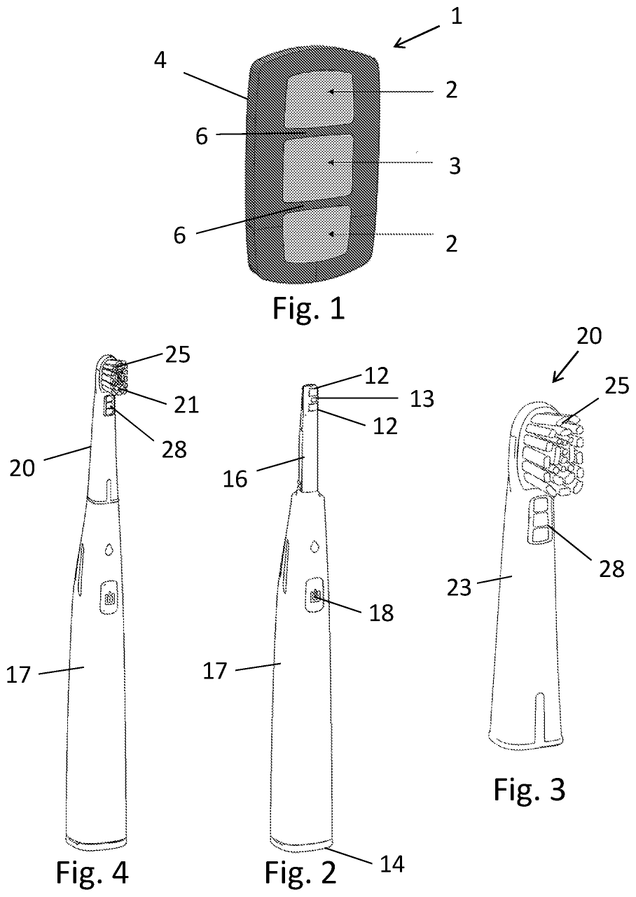

FIG. 1 is a general illustration of an imaging device used in conjunction with an intraoral device of the invention. The illustrated imaging device is particularly convenient because it can be implemented in a variety of intraoral devices such as illuminating toothbrushes and dental scanners. The imaging device of a limited width and generally indicated by numeral 1 comprises two light sources 2 and a sensor 3 interposed therebetween, wherein the light sources 2 and sensor 3 are all surrounded by a dark frame 4. Each light source 2 and sensor 3 is schematically illustrated, or alternatively is shown to be covered by a window element through which light is transmitted. Frame 4 is configured with two dividers 6 for dividing each light source from an adjacent sensor, each divider 6 being configured to obstruct or block the direct emission of light from a corresponding light source 2 to sensor 3.

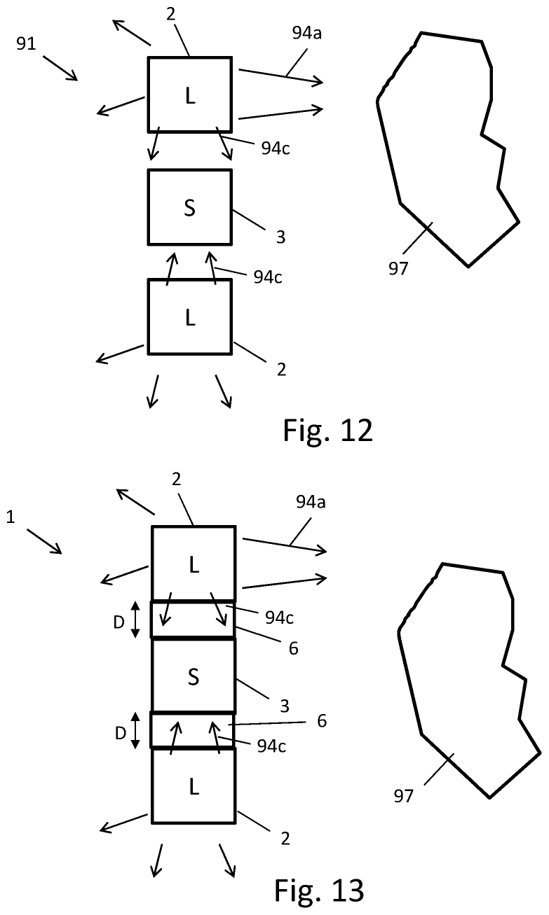

The utility of imaging device 1 will be appreciated by referring to FIGS. 12 and 13. FIG. 12 illustrates an imaging device 91 provided without a frame. The two light sources 2 are omnidirectional, meaning that the light rays emitted therefrom propagate in all directions, although the intensity of the light may be greater in a specific angular envelope, e.g. of 120 degrees, directed towards the target intraoral structure 97. In addition to light rays 94a that are directed towards target intraoral structure 97, light rays 94c of the omnidirectional light also propagate directly to sensor 3. Without the obstructing means provided by imaging device 1, light rays 94c will impinge upon, and cause saturation of, sensor 3.

FIG. 13 illustrates imaging device 1. The presence of opaque divider 6 prevents light rays 97c from propagating directly to sensor 3. The opaque divider, which has a substantially uniform width, absorbs most of the radiation of light rays 97c and occupies the entire interspace between each light source and the adjacent sensor 3. The radiation that is not absorbed by frame 4 is reflected in a direction away from sensor 3 or is converted into heat. Divider 6 may be made of a material or color that is opaque, for example a black opaque color or a white opaque color.

Based on studies conducted by the Applicant, the distance D between each light source 2 and sensor 3 ranges from 0.3-1.5 mm, for example approximately 0.75 mm, such as 0.3-0.9 mm. This distance corresponds to the maximum distance that is achievable without having the imaging device being subjected to a substantial reduction in irradiance or in uniformity of reflected light. A typical light source may be the NSSU123T LED manufactured by Nichia Corporation, Tokushima, Japan, which is an UV LED having a maximum radiant flux of 27.2 mW. Other light sources of course are suitable insofar as the generated light is not injurious to the human body when used to irradiate an intraoral structure.

Divider 6 need not be opaque, but rather made of a light absorbing material, color or coating that is capable of absorbing at least 50%, for example at least 70%, 80% or 95% of the radiation of light rays 97c, and may be made of a material that is applied to a base surface or that is surface treated. The light absorbing material may be made of different plastic materials such as polypropenyl or acrylonitrile butadiene styrene (ABS) compounds such as pigments and dyes, an organic light-absorbing material, or a material having a light absorption band with an absorption peak.

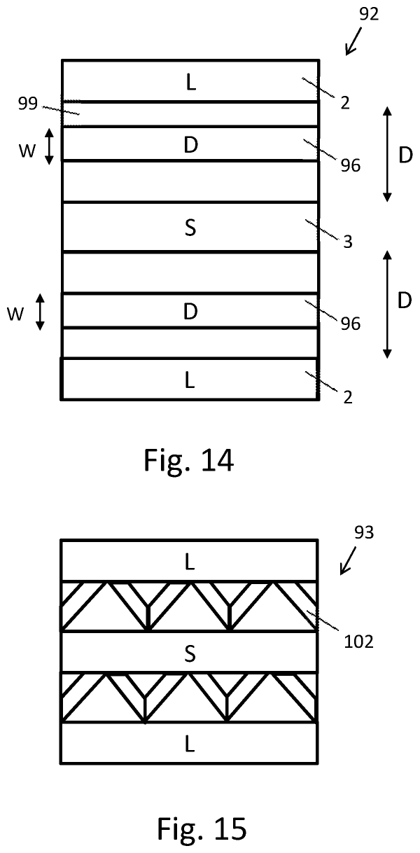

As shown in FIG. 14, an imaging device 92 may be configured with a divider 96 whose width W is less than the distance D between each light source 2 and sensor 3. The frame may be provided with a surface located within the interspace 99 between divider 96 and light source 2. Despite its small width, the light absorbing material from which divider 96 is made is sufficient to obstruct the direct emission of light from light source 2 to sensor 3.

As shown in FIG. 15, an imaging device 93 may be configured with a divider 102 having a varying orientation, such as the illustrated zig-zag configuration, or a varying width.

In the embodiment illustrated in FIG. 2, the two light sources 12 and sensor 13 of imaging device 1 are mounted at the top of a fixed post 16 protruding upwardly from a thicker and elongated handle 17, within which is housed circuitry for operating both the imaging device and the intraoral device, if different circuits are provided. A divider for blocking or obstructing light emitted from a light source 12 may be mounted to post 16. If so desired, a frame including a divider may be mounted to post 16. An activation device 18 operatively connected to handle 17 is used for activating the circuitry.

The configuration of imaging device 1 by which a single light source 12 and a single sensor 13 occupies its entire limited width at any given lengthwise region is well suited for mounting on post 16, which is also of a limited width. For example, as shown in FIG. 2, both the upper light source 12 and the lower light source 12 occupy the entire limited width of imaging device 1.

Handle 17 is adapted to be coupled with different interchangeable heads, for example toothbrush head 20 provided with a toothbrush 25 illustrated in FIG. 4, or alternatively another head used for operating a different intraoral device. A bottom portion of handle 17 distant from head 20 may be provided with a battery cap 14, which is removable in order to replace a battery. The interchangeable head has a hollow sheath 23 within which post 16 of a base member of the intraoral device is insertable. Window assembly 28 is provided within sheath 23, and is adapted to cooperate with light sources 12 and sensor 13, to obstruct the direct emission of light from each of light sources 12 to the adjacent sensor 13.

To prevent the light emitted from light sources 12, which are mounted on post 16, from impinging upon frame portions of window assembly 28, which are fixed within sheath 23, and being reflected onto sensor 13, window assembly 28 may be configured such that the divider of window assembly 28 is aligned with the divider of post 16. Also, if used, the frame of post 16 is aligned with the frame of window assembly 28.

Alternatively, sheath 23 may be configured to ensure that the distance between light sources 12 and window assembly 28 will be limited, for example less than 5 mm, e.g. 3 mm.

Consequently, even if light were reflected from frame portions of window assembly 28, the angle of reflection would be sufficiently small to prevent the reflected light from impinging upon sensor 13.

An assembled intraoral device 30 comprising toothbrush head 20 coupled with handle 17 is shown in FIG. 4.

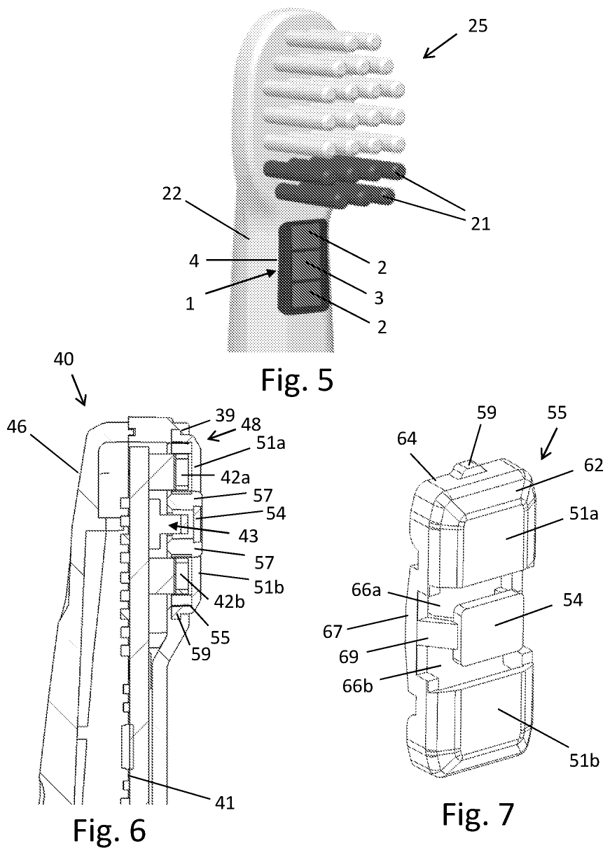

The light emitted from each light source 12 is liable to impinge upon the bristles of toothbrush head 20, reflect therefrom and be transmitted to sensor 13, often leading to saturation of the sensor. As additional means for preventing transmission of reflected light to sensor 13, according to an embodiment, at least two proximate bristle rows 21 of toothbrush 25, i.e. the rows that are closer to window assembly 28, are configured with opaque bristles, as further shown in FIG. 5. Toothbrush 25 may be part of an interchangeable head, or, alternatively, may be a dedicated single-use device that is not interchangeable, wherein imaging device 1 is mounted at a top portion 22 of handle 19 that is proximate to the bristles.

A schematic cross sectional view of an intraoral device 40 comprising a post 46, to which is coupled a window assembly 48 by connecting apparatus 39, is shown in FIG. 6. Post 46 has a non-uniform thickness, wherein an upper portion thereof is thicker than a lower portion; however, a uniformly thick post is also within the scope of the invention. A printed circuit board (PCB) 41 extends vertically substantially throughout post 46, and is in electrical communication with the two light sources 42a and 42b, shown to be LEDs, and with sensor 43. PCB 41, which may also be opaque, is configured with dedicated apparatus supporting, or connected to, the two light sources 42a-b, to ensure that their outer light emitting surface is substantially coplanar with each other and with the outer receiving surface of sensor 43, i.e. separated by no more than 0.5 mm, e.g. a separation distance of 0.1 mm or even of 0.05 mm.

Window assembly 48 comprises transparent window elements 51a-b and 54 for covering light sources 42a-b and sensor 43, respectively, as protection against possible damage resulting from contact with saliva or other liquids located within the oral cavity, while permitting light transmission. Window elements 51a-b and 54 may be made for example of polymethyl methacrylate (PMMA), acrylic or topaz, and provided with a coating. A frame 55, to which window elements 51a-b and 54 are connected or with which they are integrally formed, is coupled by oppositely protruding snap elements 59 with corresponding connecting apparatus 39 of post 46. Frame 55 may be made from the same material as window elements 51a-b and 54, or from a different material. A corresponding opaque divider 57 for obstructing the direct emission of light to sensor 43 is positioned, e.g. sealingly positioned, within the void region between window elements 51a and 54 and between window elements 51b and 54.

FIG. 7 illustrates frame 55 used in conjunction with window assembly 48 of FIG. 6, when separated from the post and shown without the dividers. In this embodiment, window elements 51a-b and 54 are integrally formed with frame 55. A corresponding transitional element 62 surrounding window elements 51a-b interfaces with peripheral element 64 from which protrudes a snap element 59. The two peripheral elements 64 are interconnected by two laterally spaced, elongated extenders 67, and window element 54 is integrated with each extender 67 by a corresponding laterally extending element 69. Two void regions 66a and 66b are thereby defined.

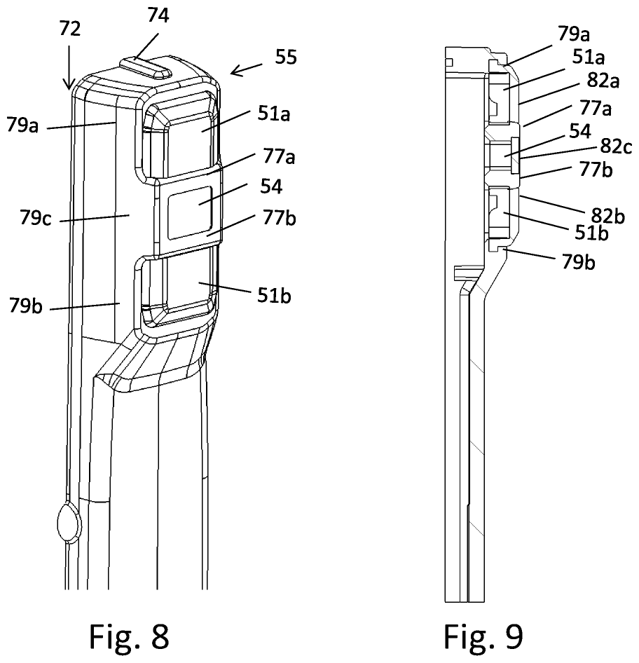

FIGS. 8 and 9 illustrate a divider mount 72, which is securable to the post, and is configured with two spaced dividers 77a and 77b that occlude void regions 66a and 66b, respectively, shown in FIG. 7. Divider mount 72 has a unitary different-sided peripheral structure 74 that overlies the two peripheral elements 64 and the two extenders 67 of frame 55. Peripheral structure 74 has three apertures 79a-c of specific dimensions that enable window elements 51a-b and 54, respectively, to be completely unobstructed by the peripheral structure material. Dividers 77a and 77b project forwardly, i.e. towards the target-facing optical plane, from an edge of apertures 79a and 79b, respectively, such that their forward surface is substantially coplanar with the optical plane 82a-c of window elements 51a-b and 54, respectively, allowing them to obstruct the direct emission of light from window elements 51a-b to window element 54.

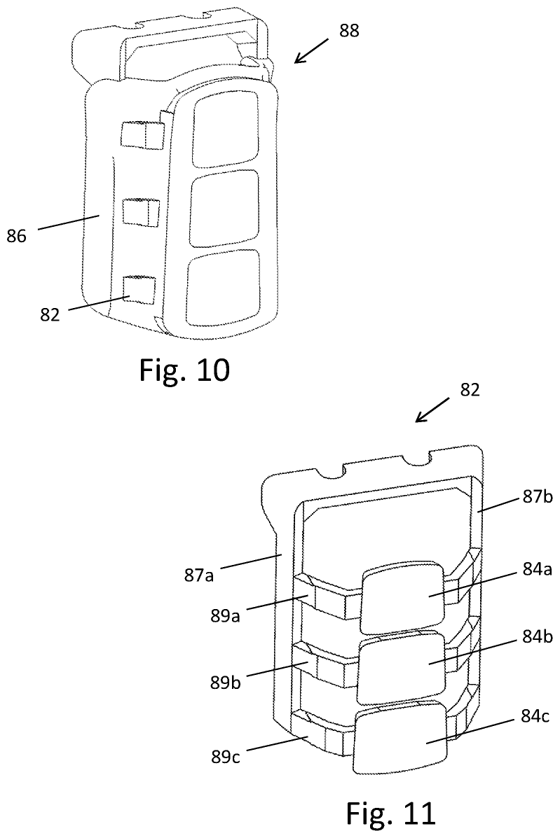

FIG. 10 illustrates a differently configured window assembly 88 comprising frame 82 and divider mount 86, including the mechanical overlays and structure. As shown in FIG. 11, frame 82 has three window elements 84a-c which are integrated with each extender 87a-b by a corresponding laterally extending element 89a-c.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1. An intraoral imaging device, comprising:
a) an elongated structural element;
b) one or more light sources mounted on said structural element and configured to illuminate a target intraoral structure;
c) one or more sensors mounted on said structural element and configured to detect the light returned from the illuminated target intraoral structure in order to generate an image; and
d) a light absorbing divider dividing each of said light sources from an adjacent sensor and configured to obstruct direct emission of the light from said one or more light sources to said adjacent sensor.

2. The intraoral imaging device according to claim 1, wherein each of the light sources is mounted at a different lengthwise region of the structural element.

3. The intraoral imaging device according to claim 1, wherein a target-facing optical plane of each of the light sources and of each of the sensors are substantially coplanar.

4. The intraoral imaging device according to claim 3, further comprising a window assembly which is configured with a plurality of protective transparent window elements for covering each of the light sources and sensors, each of said window elements constituting the target-facing optical plane.

5. The intraoral imaging device according to claim 4, wherein the window assembly is formed with a thickened portion to ensure that the target-facing optical plane of each of the light sources and of each of the sensors are substantially coplanar even though a forward surface of one of the light sources and one of the sensors while mounted on the structural element is forwardly spaced from one another.

6. The intraoral imaging device according to claim 4, further comprising a divider mount which is configured to position the divider in a void region between a first window element covering one of the light sources and a second window element which is adjacent to the first window element and covers one of the sensors.

7. The intraoral imaging device according to claim 3, wherein the window assembly is mounted on the structural element.

8. The intraoral imaging device according to claim 6, further comprising an interchangeable head with which the window assembly is fitted and within which the structural element is insertable.

9. The intraoral imaging device according to claim 8, wherein an additional divider dividing one of the light sources from the adjacent sensor is mounted on the structural element.

10. The intraoral imaging device according to claim 9, wherein, when the interchangeable head is coupled with a base member of the intraoral device, the structural element mounted divider is aligned with the head mounted divider to prevent light emitted by one of the light sources from being reflected from the head mounted divider onto the sensor.

11. The intraoral imaging device according to claim 8, wherein the interchangeable head is a toothbrush head having a toothbrush provided with at least some light absorbing bristles to prevent reflection of the light onto one of the sensors.

12. The intraoral imaging device according to claim 1, wherein the divider is opaque and is configured to completely block direct emission of the light from the one or more light sources to the adjacent sensor.

13. The intraoral imaging device according to claim 1, wherein a distance between each of the light sources and the adjacent sensor ranges from 0.3 to 1.5 mm.
